(19) United States
(12) Patent Application Publication
(10) Pub. No.: US 2007/0259939 A1
(43) Pub. Date: Nov. 8, 2007

(54) USING NALTREXONE AS A MULTI-PURPOSE HEALTH SUPPLEMENT TO IMPROVE THE HUMAN CONDITION AND PREVENTING MULTIPLE DISEASES AND INFIRMITIES BY STIMULATING IMMUNE SYSTEM VITALITY AND ROBUSTNESS

(75) Inventor: Franklin Leroy Stebbing, Norfolk, NE (US)

Correspondence Address: BANNER & WITCOFF, LTD., TEN SOUTH WACKER DRIVE, SUITE 3000, CHICAGO, IL, 60606, US

(73) Assignee: ACCELERATED TECHNOLOGIES, Norfolk, NE (US)

(21) Appl. No.: 11/550,742

(22) Filed: Oct. 18, 2006

Related U.S. Application Data
(60) Provisional application No. 60/797,587, filed on May 4, 2006.

(51) Int. Cl. A61K 31/485

(52) U.S. Cl. 514/410

(57) ABSTRACT

Naltrexone has been used to fight existing infections, afflictions, and substance abuse. Embodiments of the current invention provide naltrexone as a preventative medicine taken daily, usually in the late evening for greater efficacy. It is taken in a low dose like a vitamin supplement and may prevent many diseases by stimulating the immune system. The increased endorphin levels resulting from the use of low dose naltrexone may also reduce the need for pain relief medications, plus counter tendencies toward experiencing undesirable moods, such as depression or anger. Increased feelings of well-being produced by naltrexone may also have far-reaching effects, as suggested by studies showing that people with positive attitudes recover more quickly from serious illnesses, for example. In another aspect of the invention, naltrexone may be used to prevent substance addiction before it ever starts, by blocking the internal positive response that normally results from the use of chemical substances.

### CROSS REFERENCE TO RELATED APPLICATIONS

This application claims priority to U.S. Provisional Application No. 60/797,587 filed May 4, 2006.

### FIELD OF THE INVENTION

The invention relates to health supplements, in particular supplements for the prevention of diseases and infirmities and for the reduction of susceptibility towards chemical addiction.

### BACKGROUND OF THE RELEVANT ART

Traditional medicine has long been focused on the cure of afflictions after they are recognized by the onset of symptoms. Aspects of this invention are dedicated to the proposition that it is better to prevent diseases then to cure them after they have become established. There are numerous health supplements and vitamins available now that are just for the maintenance and/or enhancement of good health and vitality. More and more people are desirous of holding onto vigor and vitality as they age, such as “baby boomers”. Many people are determined to maintain their health and vitality for as long as it is possible to do so, and infirmity is often not accepted as inevitable.

Naltrexone has been used in low dose form since at least the mid 1980's to cure or stabilize a growing list of ailments. It is used for the treatment of a number of serious diseases, such as some cancers, HIV, AIDS and multiple sclerosis. Studies have shown naltrexone to have a positive effect in as many as 65% of cancer patients, for example stopping the spread of tumors, as well as causing remission in 30% of the patients. Low dose naltrexone functions by blocking opioid, or endorphin, receptors in various types of cells. By blocking opioid receptors, naltrexone also blocks the reception of the opioid hormones that our pituitary and adrenal glands produce: beta-endorphin and metenkephalin. Many body tissues have receptors for these endorphins and enkephalins, including virtually every cell of the body's immune system. When the receptors are blocked, the body signals an increase in endorphin production and the level of endorphins rises in reaction. An increase in endorphins causes the immune system to become stimulated and therefore more effective at fighting infection. The increased endorphin level may also dull or kill pain, and often results in a feeling of well-being because endorphins are natural mood elevating chemicals in the brain. In contrast, stress may cause a marked reduction in endorphins, which often does not correct itself naturally, thus resulting in lowered immune defenses susceptible to a variety of illnesses, such as autoimmune diseases or cancer. Cancer has occasionally been observed in patients after about two to six years following a period of extreme stress.

With respect to the treatment of alcohol addiction, naltrexone may be effective because it eliminates the neural positive reinforcement normally experienced in the brain by blocking the receptors that provide the stimulatory effect felt from alcohol. As a result of the lack of positive reinforcement, there is no internal incentive to drink alcohol. Naltrexone is not known to have any important side effects at low dose levels. The FDA approved dosage size for treatment of alcohol dependency is a 50 mg tablet, once daily. While taking naltrexone at this dosage level, it is suggested that a counselor should be involved for support and advice and supervision. It also can be given in a slow release form by injection or surgical implant if the patient is incapable or cannot be depended upon to take the medicine regularly.

### SUMMARY OF THE INVENTION

Previously, naltrexone had only been used to fight existing infections or afflictions, or to prevent recurrence. In contrast, embodiments of this invention provide naltrexone as a preventative medicine taken daily, usually in the late evening for greater efficacy. It is taken like a vitamin supplement and may prevent many of the diseases that tend to be susceptible to a strong immune system.

The increased endorphin levels resulting from the use of low dose naltrexone may also reduce the need for pain relief medications as well as counter tendencies toward experiencing undesirable moods, such as excessive depression or anger. Increased feelings of well-being may also have far-reaching effects, as suggested by studies showing that people with positive attitudes recover more quickly from serious illnesses, for example. In addition, any prevention of sickness would have the additional benefits of decreasing the use of medications with unwanted side effects and lowering health care costs.

In aspects of the invention, naltrexone may be used to prevent substance addiction before it ever starts, by taking it daily. This could result in great personal and financial savings by reducing the occurrence of addiction and thus the need for rehabilitation programs. Unlike existing medications such as Antabuse, a drug that will induce nausea or even unconsciousness if it is taken followed by alcohol consumption, naltrexone is more effective when taken prior to drinking alcohol because it reduces or removes the desire to drink.

### DETAILED DESCRIPTION OF EMBODIMENTS OF THE INVENTION

One embodiment of the invention is a method of using low doses of naltrexone to block endorphin receptors in various body cells of a human or animal patient. When the receptors are blocked, the body signals an increase in endorphin production and the level of endorphins rises in reaction. The method may comprise a daily dose of naltrexone administered to the patient, for example between approximately 0.5 and 5 milligrams of naltrexone, and preferably between 1.75 and 4.5 milligrams of naltrexone. An aspect of the invention provides administration of the daily dose of naltrexone in the late evening hours, such as between 6:00 pm and 2:00 am, so as to block the opioid receptors at a time when endorphins are naturally produced by the body's circadian rhythm, usually in the early morning between 2:00 am and 4:00 am, regardless of wakefulness or sleep. The brief blockade of opioid receptors between 2 a.m. and 4 a.m. may produce a prolonged stimulation of the immune system by causing an increase in endorphin and enkephalin production. In fact, tests have shown that taking small doses of naltrexone each night has resulted in much higher levels of beta-endorphins circulating in the blood in the following days, providing as much as a 200-300% increase in patients who initially had deficient endorphin levels. The naltrexone may be taken as a tablet, capsule or diluted in liquid, as long as it is present in a readily bioavailable form.

An embodiment of the invention is a method to stimulate the immune system of a patient prior to the onset of an illness via a daily dose of naltrexone that is generally in the range of 0.5 to 5 milligrams per dose, and again preferably between about 1.75 and 4.5 milligrams per dose. The resulting increase in endorphins thus causes a stimulation of immune system function with marked improvement in disease fighting capability. In an aspect of the invention, the administration of a daily dose of naltrexone, for example in the late evening, may prevent the onset of an illness in a human or animal patient. The method includes taking low doses of naltrexone prior to potentially being exposed to infectious conditions, with or without additional medications or vaccinations to cause antibody production. An increase in endorphins resulting from the blockage of endorphin receptors by the naltrexone causes the immune system to become more effective at fighting infection during its early stages. The use of naltrexone as taught here may improve immune system function, resulting in a greatly enhanced ability to fend off debilitating diseases such as many forms of cancer, AIDS, HIV, multiple sclerosis, ALS, Alzheimer's Disease, chronic fatigue syndrome, emphysema, Parkinson's Disease, primary lateral sclerosis, psoriasis, rheumatoid arthritis, sarcoidosis and systemic lupus. For example, cancer cells are routinely produced in the body, but a healthy immune system generally kills them as they occur. In fact, endorphins added to growing human cancerous tissue in a laboratory Petri dish have been observed to kill the cancerous tissue. In contrast to waiting for a disease to become established and then taking steps to cure it with medications like naltrexone and/or other means such as surgery or chemotherapy, the disease may be prevented. Additionally, if an illness does become established, the daily naltrexone therapy may reduce or eliminate the need for various medications to overcome the illness because of the enhanced natural ability of the immune system.

An additional aspect of the invention is a method to alleviate or reduce pain in a human or animal patient comprising a daily administration of low dose naltrexone. A naltrexone dose of between about 0.5 and 5 milligrams may be used, for example taken during the late evening such as from 6:00 pm to 2:00 am, as described above. The increased endorphin level may dull or kill pain, as endorphin levels naturally increase when injury occurs. Because naltrexone stimulates an endorphin increase, naltrexone therapy may reduce aches and pains.

The employment of a daily low dose of naltrexone may prevent one or more psychological problems in a human, in an embodiment of the invention. This is due to the resulting increase of endorphins, which are natural mood elevating chemicals in the brain. An increased level of endorphins may preclude or prevent the onset of some mental depression or anger, for example, because endorphins are natural anti-depressives and mood enhancers. As a result of the absence of depression and improved mood and feeling of well-being, naltrexone may reduce anti-social behavior with more incidences of positive social interactions.

Naltrexone is already known to help cure addictive behaviors, such as alcoholism, opiate, narcotic and tobacco addiction. With respect to the treatment of alcohol addiction, naltrexone may be effective because it eliminates the neural positive reinforcement normally experienced in the brain by blocking the receptors that provide the stimulatory effect felt from alcohol. As a result of the lack of positive reinforcement, there is no internal incentive to drink alcohol. In a similar manner, in another embodiment of the invention, the daily administration of low dose naltrexone may greatly reduce or eliminate the tendency to engage in addictive behavior by preventing its onset. This may be accomplished by diminishing any potential internal rewards for using chemical substances in the first place. The low dosage of naltrexone, for example around 4.5 mg, has been found to greatly inhibit alcohol cravings. The diminished desire for alcohol resulting from a low dose of naltrexone may be effective for more than 24 hours, thus for example a dose administered at 7:00 pm one evening may continue to inhibit alcohol cravings throughout the following evening. A person in need of curbing an alcoholic appetite may take a pill, either before or along with the drink, and discover no desire for a second or third drink. To further eliminate an alcoholic appetite, a series of 1.5 mg naltrexone doses may be taken without alcohol, starting for example at about 6:00 pm. Three doses, for a total of 4.5 mg naltrexone, may be taken, for example two or three doses together or single doses over a period of time, such as one per hour, as provides the most effective result for the individual.

It is known that it is very important to have a positive attitude and outlook when battling and treating serious illnesses, such as cancer. The outcome is much better if the patient is upbeat and optimistic. This is similar to what happens to those who take low doses of naltrexone to boost endorphin levels to combat diseases. The low doses of naltrexone raise the level of endorphins, which eventually leads to an increase in immune system function. Either way, naturally or naltrexone induced, the increased levels of endorphins result in a better mood and an improved, enhanced and more robust immune system function, which equates to an improved prognosis due to a greater ability of the immune system to overcome and defeat a disease. If a patient does not have a naturally optimistic attitude, naltrexone may help provide a good disposition with the same beneficial results, by artificially stimulating the immune system.

Variations and modifications of the foregoing are within the scope of the present invention. It should be understood that the invention disclosed and defined herein extends to all alternative combinations of two or more of the individual features mentioned or evident from the text. All of these different combinations constitute various alternative aspects of the present invention. The embodiments described herein explain the best modes known for practicing the invention and will enable others skilled in the art to utilize the invention. The claims are to be construed to include alternative embodiments to the extent permitted by the prior art. For example, endorphins may be administered directly without the use of naltrexone such as in those cases where a patient is not able to produce endorphins by naltrexone stimulation, or where the use of naltrexone is prevented by other factors. For instance, because naltrexone is an opiate agonist, it may not be appropriate to administer naltrexone to a patient using opiates for pain medication.

Various features of the invention are set forth in the following claims.

What is claimed is:

1. A method to increase endorphin levels in a human or animal patient comprising a daily administration of a dose of between about 0.5 mg and about 5 mg naltrexone.

2. The method according to claim 1 wherein the naltrexone is administered to the patient during an evening hour.

3. The method according to claim 2 wherein the evening hour is between approximately 6:00 pm and approximately 2:00 am.

4. A method to stimulate the immune system of a human or animal patient prior to the onset of an illness comprising a daily administration of a dose of naltrexone.

5. The method according to claim 4 wherein the dose of naltrexone is between about 0.5 mg and about 5 mg.

6. The method according to claim 4 wherein the naltrexone is administered to the patient during an evening hour.

7. The method according to claim 6 wherein the evening hour is between approximately 6:00 pm and approximately 2:00 am.

8. A method to prevent addiction of a human or an animal patient to a chemical substance comprising a daily administration of a dose of naltrexone.

9. The method according to claim 8 wherein the naltrexone is administered prior to any use of the chemical substance.

10. The method according to claim 8 wherein the dose of naltrexone is between about 0.5 mg and about 5 mg.

11. The method according to claim 9 wherein the chemical substance is an opiate.

12. The method according to claim 9 wherein the chemical substance is alcohol.

13. The method according to claim 9 wherein the chemical substance is nicotine.

14. A method to reduce physical pain in a human or animal patient comprising a daily administration of a dose of between about 0.5 mg and about 5 mg naltrexone.

15. The method of claim 14 wherein the naltrexone is administered to the patient during an evening hour.

16. The method of claim 15 wherein the evening hour is between approximately 6:00 pm and approximately 2:00 am.

17. A method to prevent the onset of illness in a human or animal patient comprising a daily administration during an evening hour of a dose of between about 0.5 mg and about 5 mg naltrexone.

18. The method of claim 17 wherein the evening hour is between approximately 6:00 pm and approximately 2:00 am.

19. A method to prevent one or more psychological problems in a human patient comprising a daily administration of a dose of between about 0.5 mg and about 5 mg naltrexone.

20. The method of claim 19 wherein the one or more psychological problems are selected from the group comprising depression, anger and anti-social behavior.
